(19) United States
(12) Patent Application Publication
(10) Pub. No.: US 2017/0333245 A1
(43) Pub. Date: Nov. 23, 2017

(54) MULTI-FUNCTIONAL DISPOSABLE MALE URINE GUIDING DEVICE

(71) Applicant: Xiao Ming Shi, Guangdong (CN)

(72) Inventor: Xiao Ming Shi, Guangdong (CN)

(21) Appl. No.: 15/186,904

(22) Filed: Jun. 20, 2016

(30) Foreign Application Priority Data
May 23, 2016 (CN) 201610337948.5

(51) Int. Cl. A61F 5/453; A47K 11/12

(52) U.S. Cl. CPC A61F 5/453; A47K 11/12

(57) ABSTRACT

A funnel-shaped paper urine guiding device is provided having sufficient strength and waterproof ability and capable of being flushed down the toilet without plugging sewer systems. The urine guiding device comprises a penis inlet, a urine outlet, and a handheld protruding flap, and is capable of eliminating the splashing of urine on the toilet perimeter or in the surrounding area by a man urinating from a standing position. The urine guiding device is able to minimize the amount of splashed urine and be used to wipe the residual urine off the male user's penis after urination. Therefore, the urine guiding device of the present invention facilitates urination by men from a standing position. The device is advantageous for men's physical-mental health and makes the cleaning of the toilet facilities easier, thereby reducing family conflicts. Furthermore, the device is easy to use and inexpensive.

### CROSS-REFERENCE TO RELATED APPLICATION(S)

This application claims priority under 35 U.S.C. §119 of the Chinese Patent Application Ser. No. 201610337948.5, filed May 23, 2016 and titled, “Multi-Functional Disposable Male Urine Guiding Device,” which is also hereby incorporated by reference in its entirety for all purposes.

### FIELD OF THE INVENTION

The present invention relates to a disposable multifunctional sanitary aid, which can be used to direct the urine stream of a male user urinating from a standing position to the toilet and wipe the residual urine off the penis of the male user before being flushed down the toilet.

### BACKGROUND OF THE INVENTION

From the medical aspect, the male has a unique organ-prostate. Urinating from a standing position can ease an excess of blood in the pelvic cavity under the pressure caused by long periods of sitting time so that urine can be fully discharged, significantly reducing the risks of prostatitis and lower urinary tract obstruction. Some men have difficulty controlling the speed and direction of their urine streams when urinating from a standing position, thus the urine streams often miss the toilet bowl completely, landing either on the toilet perimeter or in the surrounding area, leaving the toilet in a soiled condition. The splashes of urine must be periodically cleaned up to prevent the unpleasant odor, requiring a lot more cleaning work to be done. The soiling of the toilet can also be a cause of family conflicts. Therefore, some men choose to urinate from a sitting position to avoid such conflicts. However, urinating from a sitting position is bad for men's health. For some men with longer penises, they are likely to wet their underpants and splash urine beyond the toilet bowl even if they urinate from a sitting position. Moreover, the residual urine on the penis leaves stains on the underpants, which is not sanitary and can lead to an unpleasant odor as well.

### SUMMARY OF THE INVENTION

To solve the aforementioned problem, a disposable multifunctional urine guiding device is provided, which is of simple construction, convenient and inexpensive and facilitates urination by men from a standing position.

In some embodiments, a disposable urine guiding device disclosed herein comprises: a penis inlet configured to receive the penis of a male user of the urine guiding device therein; a urine outlet configured to discharge urine into a toilet bowl; and a handheld protruding flap configured such that the male user can hold the urine guiding device; wherein the urine guiding device is made of a paper material and formed into a funnel shape.

In some embodiments, the urine guiding device is folded into the funnel shape from at least one sheet of paper. The at least one sheet of paper has a thickness ranging from 0.01 to 1 mm.

In some embodiments, the urine guiding device comprises an overlapping portion having a thickness more than twice the thickness of the at least one sheet of paper. The penis inlet is positioned opposite to the overlapping portion. The handheld protruding flap is positioned at the same side as the overlapping portion and is an integral part of the overlapping portion.

In some embodiments, the penis inlet has a short axis with a length ranging from 3 to 30 cm.

In some embodiments, the urine outlet has a short axis with a length greater than 0.2 cm.

In some embodiments, the handheld protruding flap has a length and a width each of which ranges from 0.5 cm to 10 cm.

In some embodiments, the urine guiding device has a height ranging from 5 cm to 30 cm.

The features and advantages of the present invention will be detailed in the following preferred embodiment so that those skilled in the art can understand the technical content of the present invention and thereby to practice the present invention. Moreover, those skilled in the art can easily understand the objects and advantages of the present invention according to the disclosure in the description, claims and drawings.

### BRIEF DESCRIPTION OF THE DRAWINGS

FIGS. 1-3 are schematic views illustrating a process of manufacturing a urine guiding device in accordance with some embodiments.

FIG. 4 is a section view of the urine guiding device in accordance with some embodiments.

FIG. 5 is a schematic view illustrating a male user using the urine guiding device in accordance with some embodiments.

### DETAILED DESCRIPTION OF THE PREFERRED EMBODIMENT

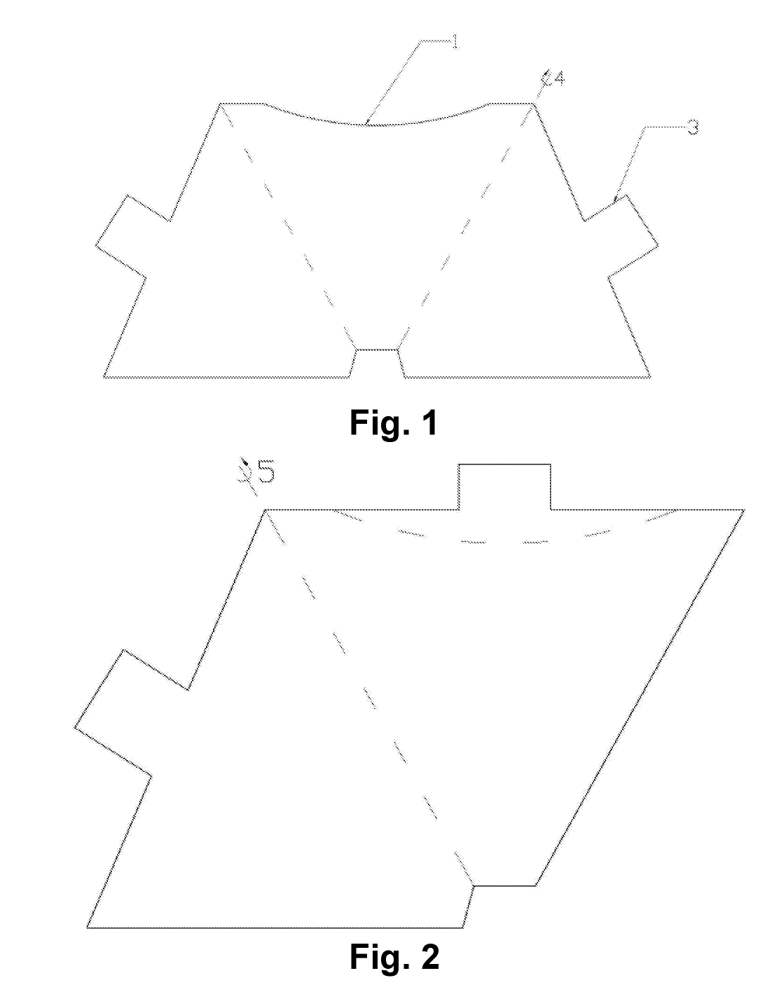

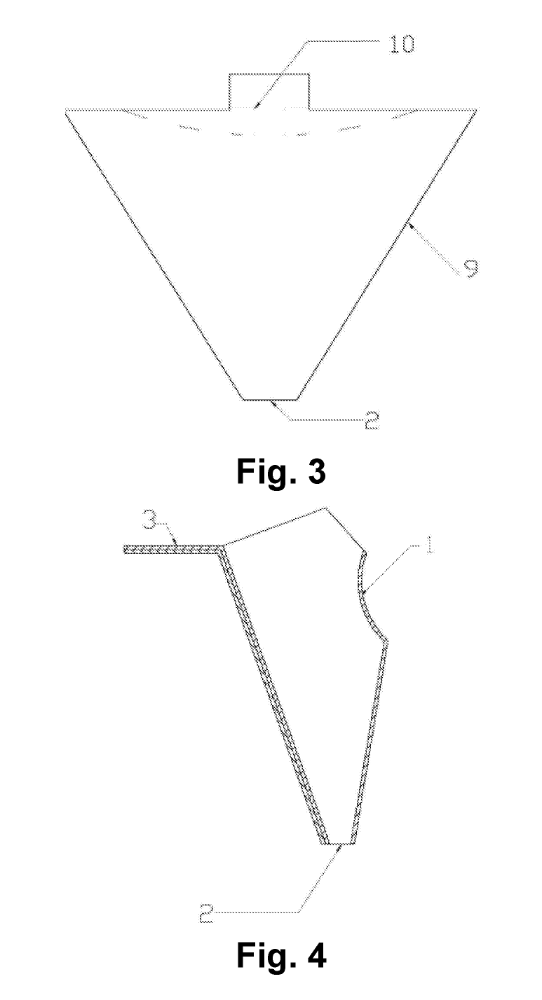

FIGS. 1-3 are schematic views illustrating a process of manufacturing a urine guiding device in accordance with some embodiments. The urine guiding device is made of one (or multiple superimposed) sheet(s) of shaped paper, as shown in FIG. 1. In some embodiments, the sheet of paper has a thickness ranging from 0.01 to 1 mm. First, the sheet of paper is folded in a direction indicated by an arrow shown in FIG. 1 along a diagonal line 4 into the shape shown in FIG. 2. Next, the sheet of paper is folded in a direction indicated by an arrow shown in FIG. 2 along a diagonal line 5 into the shape shown in FIG. 3. Next, regions 9 and 10 are adhered at an overlapping portion thereof, and then both sides of the urine guiding device are slightly squeezed to form a funnel shape. Please also refer to FIG. 4. In some embodiments, the urine guiding device comprises a penis inlet 1, a urine outlet 2 and a handheld protruding flap 3. The penis inlet 1 is positioned opposite to the overlapping portion. The handheld protruding flap is positioned at the same side as the overlapping portion and is an integral part of the overlapping portion.

In some embodiments, the penis inlet 1 has a size greater than an average penis diameter so that the penis can be placed into the urine guiding device easily In some embodiments, the penis inlet 1 has a short axis with a length ranging from 3 to 30 cm. According to the urine guiding device of the present invention, the urine outlet 2 has a size greater than an average male ureteral diameter and smaller than the penis inlet 1 so that urine will be discharged in a timely manner without being retained in the urine guiding device and the amount of urine to be discharged within one unit time can be controlled to reduce the amount of splashed urine. In some embodiments, the urine outlet 2 has a short axis with a length greater than 0.2 cm. The urine guiding device has a height substantially the same as an average penis length ranging from about 5 cm to 30 cm. In some embodiments, the handheld protruding flap 3 has a size suitable to be held by all male users and can be used to wipe the residual urine off the penis after urination.

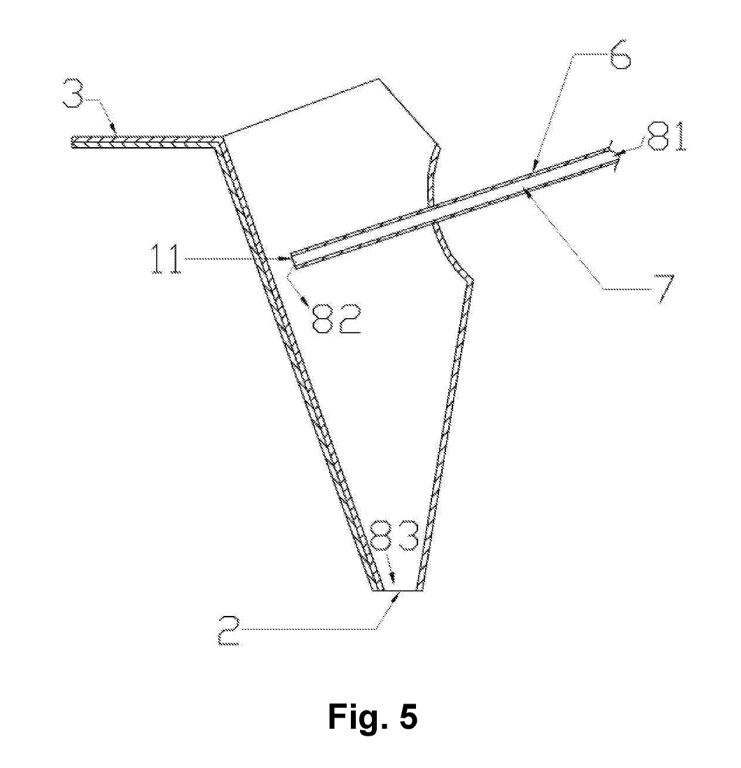

FIG. 5 is a schematic view illustrating a male user using the urine guiding device in accordance with some embodiments. When using the urine guiding device, the male user holds the handheld protruding flap 3, props open the penis inlet 1, places the penis 6 into the urine guiding device in a direction indicated in FIG. 5, and aims the urine outlet 2 at the toilet bowl before urination. Urine in the ureter 7 discharged in a direction denoted by the reference sign 81 in FIG. 5 first impinges on an inner wall of the urine guiding device, and then flows toward the urine outlet 2 along the inner wall in directions denoted by the reference signs 82 and 83. At this point, the urine stream is decelerated to substantially zero velocity, and urine is discharged to free-fall to the toilet bowl after flowing to the urine outlet 2. As the inner wall is the overlapping portion of the region 9 and the region 10, the thickness thereof is twice the thickness of the sheet of paper so as to ensure that the sheet of paper has sufficient strength not to break when impinged by the urine stream. In the preferred embodiment of the present invention, the sheet of paper has a thickness ranging from 0.01 to 1 mm.

After the male user is done urinating with the urine guiding device of the present invention, he can wipe the residual urine off the glans 11 of the penis and flush it down the toilet. The urine guiding device ensures that urine does not be splashed on the toilet perimeter or in the surrounding area. As the urine stream is baffled by the inner wall of the urine guiding device and the velocity thereof is substantially decelerated to zero before urine is discharged to free-fall to the toilet bowl, the amount of urine splashed on the toilet perimeter is significantly reduced, thereby maintaining the cleanliness of the toilet facilities. The use of the handheld protruding flap 3 to wipe the residual urine off the penis also prevents the underpants from being stained by urine.

In utilization, the urine guiding device is able to be used to guide urine of a male user.

In operation, the male user holds the handheld protruding flap, props open the penis inlet, places the penis into the urine guiding device in a direction, and aims the urine outlet at the toilet bowl before urination.

Although some embodiments have been shown and described above, the present invention is not limited thereto. It will be apparent to those skilled in the art that a number of changes and modifications made without departing from the spirit of the present invention shall be within the scope of the present invention. Therefore, the scope of protection of the present invention shall be construed based on the appended claims.

1. A disposable urine guiding device comprising:
a penis inlet configured to receive the penis of a male user of the urine guiding device therein;
a urine outlet configured to discharge urine to a toilet bowl, wherein the penis inlet is unparallel to the urine outlet;
a handheld protruding flap configured such that the male user can hold the urine guiding device; and
wherein the urine guiding device is made of a paper material and formed into a funnel shape.

2. The urine guiding device according to claim 1, wherein the urine guiding device is folded into the funnel shape from at least one sheet of paper.

3. The urine guiding device according to claim 2, wherein the at least one sheet of paper has a thickness ranging from 0.01 to 1 mm.

4. The urine guiding device according to claim 2, wherein the urine guiding device comprises an overlapping portion having a thickness more than twice the thickness of the at least one sheet of paper.

5. The urine guiding device according to claim 4, wherein the penis inlet is positioned opposite to the overlapping portion.

6. The urine guiding device according to claim 4, wherein the handheld protruding flap is positioned at the same side as the overlapping portion.

7. The urine guiding device according to claim 4, wherein the handheld protruding flap is an integral part of the overlapping portion.

8. The urine guiding device according to claim 1, wherein the penis inlet has a short axis with a length ranging from 3 to 30 cm.

9. The urine guiding device according to claim 1, wherein the urine outlet has a short axis with a length greater than 0.2 cm.

10. The urine guiding device according to claim 1, wherein the handheld protruding flap has a length and a width each of which ranges from 0.5 cm to 10 cm.

11. The urine guiding device according to claim 1, wherein the urine guiding device has a height ranging from 5 cm to 30 cm.
